(19) United States
(12) Patent Application Publication
(10) Pub. No.: US 2009/0297467 A1
(43) Pub. Date: Dec. 3, 2009

(54) COSMETIC COMPOSITION COMPRISING A SULPHONATED POLYMER AND AN ACRYLATE OR METHACRYLATE AND ACRYLATE OR METHACRYLATE HYDROXYESTER COPOLYMER, AND THE USE THEREOF AS A HAIR CARE PRODUCT

(76) Inventors: Ludivine Laurent, Cuartres (FR); Cecile Bebot, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/281,454

(22) PCT Filed: Mar. 2, 2007
(86) PCT No.: PCT/FR2007/050874

§ 371 Date: Jun. 15, 2009

Related U.S. Application Data
(60) Provisional application No. 60/789,926, filed on Apr. 7, 2006.

(30) Foreign Application Priority Data
Mar. 3, 2006 (FR) 0601943

(51) Int. Cl. A61K 8/81; A61Q 5/06; A61Q 5/00

(52) U.S. Cl. 424/70.16; 424/78.35; 424/78.32

(57) ABSTRACT

A subject-matter of the present invention is a cosmetic composition comprising, in a cosmetically acceptable medium:
at least one copolymer of acrylate(s) or of methacrylate(s) and of hydroxyester of acrylate(s) or of methacrylate(s), and
at least one partially or completely neutralized sulphonated polymer comprising at least one unit derived from a monomer comprising ethylenic unsaturation and comprising a sulpho group,
the ratio by weight of the amount of copolymer(s) of acrylate(s) or of methacrylate(s) and of hydroxyester of acrylate(s) or of methacrylate(s), on the one hand, to the amount of sulphonated polymer(s), on the other hand, being between 0.1 and 10.

This composition is used in particular as a styling product.

The present invention relates to a cosmetic composition comprising, in a cosmetically acceptable medium, at least one sulphonated polymer and at least one copolymer of acrylate(s) or of methacrylate(s) and of hydroxyester of acrylate(s) or of methacrylate(s).

The present invention also relates to a method for the cosmetic treatment of the hair employing the abovementioned composition and to the use of this cosmetic composition as hair product, for example as styling product.

Styling products are normally used to build or structure the hairstyle and to provide it with lasting hold. The corresponding compositions generally comprise one or more film-forming agents in a cosmetically acceptable medium. These polymers make possible the formation of a sheathing film on the individual hairs, thus ensuring the form retention of the hairstyle.

However, the fixing polymer films thus formed exhibit the disadvantage of being relatively friable, which limits the hold of the hairstyle over time and results in the formation, on the hair, of unsightly residues.

It is known, in order to reduce these problems of friability, to incorporate a plasticizing agent in styling products based on film-forming polymers. This makes it possible, in fact, to improve the hold of the hairstyle over time and to limit the formation of residues during the day.

However, despite the addition of such a plasticizing agent, the nonfriability of the films of polymers is generally related to the concentration of fixing polymer in the styling product (or degree of fixing): if the concentration of fixing polymer is reduced in order to reduce the stiffness of the hairstyle, then the polymer film becomes more friable and the hold of the hairstyle is not as good.

There thus exists a need for hair compositions which make it possible to obtain lasting form retention of the hairstyle, whatever the degree of fixing.

The Applicant Company has now discovered that, surprisingly, the combination of a specific fixing polymer, of the (meth)acrylate(s)/hydroxyester of (meth)acrylate(s) type, with a sulphonated polymer according to a specific ratio by weight between the amounts of these two types of polymers makes it possible to obtain a hair composition providing improved styling properties. In particular, such a combination makes it possible to obtain styling products which provide a range from flexible to ultrastrong fixings, with a polymer film of extremely low friability, whatever the degree of fixing. It thus becomes possible to formulate products possessing long lasting flexible fixing.

The present invention makes it possible in particular to prepare styling products which provide a markedly improved durability of the hairstyle, including in flexible fixing products, that is to say having a low concentration of fixing polymer.

Furthermore, and entirely unexpectedly, the compositions according to the invention have proved to provide a very marked improvement in the cosmetic properties of the hair after disentangling: the hair is much softer and less dry than after the application of a conventional fixing product.

A subject-matter of the present invention is thus a cosmetic composition comprising, in a cosmetically acceptable medium:
at least one copolymer of acrylate(s) or of methacrylate(s) and of hydroxyester of acrylate(s) or of methacrylate(s), and
at least one partially or completely neutralized sulphonated polymer comprising at least one unit derived from a monomer comprising ethylenic unsaturation and comprising a sulpho group,
the ratio by weight of the amount of copolymer(s) of acrylate(s) or of methacrylate(s) and of hydroxyester of acrylate(s) or of methacrylate(s), on the one hand, to the amount of sulphonated polymer(s), on the other hand, being between 0.1 and 10.

Another subject-matter of the invention is a method for the cosmetic treatment of the hair employing a composition according to the invention as described below.

A further subject-matter of the invention is the use of the composition according to the invention in the cosmetic treatment of the hair, in particular for the shaping and/or the form retention of the hairstyle.

Other subject-matters, characteristics, aspects and advantages of the invention will become even more clearly apparent on reading the description and example which follow.

According to the invention, the cosmetic composition comprises, in a cosmetically acceptable medium, at least one specific sulphonated polymer and at least one copolymer of acrylate(s) or of methacrylate(s) and of hydroxyester of acrylate(s) or of methacrylate(s).

The term “cosmetically acceptable medium” is understood to mean a medium compatible with keratinous substances and in particular the hair.

Preferably, the cosmetically acceptable medium comprises water and/or at least one cosmetically acceptable solvent chosen from lower C₁-C₄ alcohols, such as ethanol, isopropanol, tert-butanol or n-butanol; polyols, such as propylene glycol; polyol ethers; C₅-C₁₀ alkanes; C₃₋₄ ketones, such as acetone and methyl ethyl ketone; C₁-C₄ alkyl acetates, such as methyl acetate, ethyl acetate and butyl acetate; dimethoxyethane or diethoxyethane; and their mixtures.

The composition according to the invention comprises at least one copolymer of acrylate(s) or of methacrylate(s) and of hydroxyester of acrylate(s) or of methacrylate(s), that is to say a copolymer obtained by copolymerization of at least two monomers:
at least one first monomer chosen from acrylic acid, methacrylic acid and C₁ to C₄ alkyl acrylates and methacrylates and
at least one second monomer chosen from C₁ to C₄ hydroxyalkyl acrylates and methylacrylates.

Preferably, the said copolymer is obtained by copolymerization of at least three monomers:
at least one first monomer chosen from acrylic acid and methacrylic acid,
at least one second monomer chosen from C₁ to C₄ alkyl acrylates and methacrylates, and
at least one third monomer chosen from C₁ to C₄ hydroxyalkyl acrylates and methacrylates.

Preferably, the said second monomer comprises at least one C₁ or C₂ alkyl acrylate or methacrylate. In other words, the said second monomer can then be methyl acrylate, ethyl acrylate, methyl methacrylate, ethyl methacrylate and the mixtures of these compounds.

Preferably, the said third monomer is chosen from hydroxyethyl acrylate, hydroxyethyl methacrylate and mixtures of these compounds.

According to a preferred embodiment, the composition according to the invention comprises a blend of at least two copolymers of acrylate(s) or of methacrylate(s) and of hydroxyester of acrylate(s) or of methacrylate(s) as described above.

It can be a blend of at least two copolymers comprising at least two monomers as described above, a blend of at least two copolymers comprising at least three monomers as described above or a blend of at least one copolymer comprising at least two monomers and of at least one copolymer comprising at least three monomers as described above.

In a first alternative form of this preferred embodiment, the composition according to the invention comprises a blend of two copolymers of acrylate(s) or of methacrylate(s) and of hydroxyester of acrylate(s) or of methacrylate(s), each of the copolymers being obtained by copolymerization of at least three monomers as described above, namely:
at least one first monomer chosen from acrylic acid and methacrylic acid,
at least one second monomer chosen from C₁ to C₄ alkyl acrylates and methacrylates, and
at least one third monomer chosen from C₁ to C₄ hydroxyalkyl acrylates and methacrylates.

In a second alternative form of this preferred embodiment, the composition according to the invention comprises a blend of two copolymers of acrylate(s) or of methacrylate(s) and of hydroxyester of acrylate(s) or of methacrylate(s) comprising a first copolymer obtained by copolymerization of at least three monomers as described above:
at least one first monomer chosen from acrylic acid and methacrylic acid,
at least one second monomer chosen from C₁ to C₄ alkyl acrylates and methacrylates, and
at least one third monomer chosen from C₁ to C₄ hydroxyalkyl acrylates and methacrylates;
   and a second copolymer obtained by copolymerization of at least two monomers:
at least one first monomer chosen from C₁ to C₄ alkyl acrylates and methacrylates, and
at least one second monomer chosen from C₁ to C₄ hydroxyalkyl acrylates and methacrylates;
   the said second copolymer not comprising monomers composed of unsaturated carboxylic acids.

When the composition according to the invention comprises a blend of at least two copolymers of acrylate(s) or of methacrylate(s) and of hydroxyester of acrylate(s) or of methacrylate(s), the two copolymers of this blend preferably have different glass transition temperatures.

The glass transition temperature (Tg) is measured by differential scanning calorimetry (DSC) according to Standard ASTM D3418-97.

One of the two copolymers preferably has a Tg ranging from 35° C. to 250° C., more preferably from 40° C. to 150° C. and more preferably still from 75° C. to 100° C. The second copolymer preferably has a Tg ranging from −20° C. to 35° C., more preferably from 0° C. to 35° C. and more preferably still from 15° C. to 30° C.

The difference in Tg between the two copolymers is preferably greater than or equal to 10° C., more preferably greater than or equal to 20° C. and more preferably still greater than or equal to 30° C.

The copolymer(s) of acrylate(s) or of methacrylate(s) and of hydroxyester of acrylate(s) or of methacrylate(s) which can be used in the compositions according to the invention can be (a) copolymer(s) of block, random or alternating type. Preferably, it is a random copolymer.

Advantageously, the copolymer of acrylate(s) or of methacrylate(s) and of hydroxyester of acrylate(s) or of methacrylate(s) is an anionic copolymer.

More advantageously, it comprises hard blocks and soft blocks, that is to say blocks having different glass transition temperatures (Tg), one of the Tg values being between 15 and 35° C., preferably between 20 and 30° C., the other Tg value between 90 and 110° C., preferably between 95 and 105° C.

Advantageously, the hard block comprises at least one monomer chosen from methyl methacrylate, butyl acrylate, hydroxyethyl methacrylate or methacrylic acid and more advantageously the hard block comprises all these monomers.

Advantageously, the soft block comprises at least one monomer chosen from ethyl acrylate, butyl acrylate, hydroxyethyl methacrylate or methacrylic acid and more advantageously the soft block comprises all these monomers.

Preferably, the molecular weight of this copolymer is between 10 000 g/mol and 200 000 g/mol. More preferably still, it is between 20 000 g/mol and 100 000 g/mol.

The copolymers of acrylate(s) or of methacrylate(s) and of hydroxyester of acrylate(s) or of methacrylate(s) can be provided in highly diverse forms, for example in the form of solutions or emulsions in water or in any other solvent.

They can be synthesized by any known method for the preparation of a copolymer from its various monomers.

Mention may be made, as example of copolymers and blends of copolymers which can be used in the compositions according to the invention, inter alia, of the products sold under the name Acudyne by Röhm & Haas, such as, for example, and without limitation, the products provided under the names Acudyne DHR, Acudyne 258 and Acudyne 180.

The composition according to the invention preferably comprises at least 0.01% by weight of copolymer(s) of acrylate(s) or of methacrylate(s) and of hydroxyester of acrylate(s) or of methacrylate(s), with respect to the total weight of the composition. Preferably, it comprises from 0.01 to 20% by weight of copolymer(s) of acrylate(s) or of methacrylate(s) and of hydroxyester of acrylate(s) or of methacrylate(s), more preferably still from 0.1 to 10% by weight and better still from 0.5 to 8% by weight, with respect to the total weight of the composition.

The composition according to the invention also comprises at least one partially or completely neutralized sulphonated polymer comprising at least one unit derived from a monomer comprising ethylenic unsaturation and comprising a sulpho group.

The term “monomer comprising ethylenic unsaturation and comprising a sulpho group” is understood to mean, within the meaning of the present invention, a monomer having at least one double bond and carrying, in its structure, an SO₃X group, with X denoting a hydrogen or an ion of an alkali metal or alkaline earth metal, an ammonium ion or also an ion resulting from an organic amine.

The monomers comprising ethylenic unsaturation and comprising a sulpho group are preferably chosen from vinylsulphonic acid, styrenesulphonic acid, (meth)acrylamido(C₁-C₂₂)alkylsulphonic acids, N—(C₁-C₂₂)alkyl(meth)acrylamido(C₁-C₂₂)alkylsulphonic acids, such as undecylacrylamidomethanesulphonic acid, and their partially or completely neutralized forms.

More preferably, the monomers comprising ethylenic unsaturation are chosen from (meth)acrylamido(C₁-C₂₂)alkylsulphonic acids, such as, for example, acrylamidomethanesulphonic acid, acrylamidoethanesulphonic acid, acrylamidopropanesulphonic acid, 2-acrylamido-2-methylpropanesulphonic acid, methacrylamido-2-methylpropanesulphonic acid, 2-acrylamido(n-butane)sulphonic acid, 2-acrylamido-2,4,4-trimethylpentanesulphonic acid, 2-methacrylamidododecylsulphonic acid, 2-acrylamido-2,6-dimethyl-3-heptane-sulphonic acid and their partially or completely neutralized forms.

Use can advantageously be made of 2-acrylamido-2-methylpropanesulphonic acid (AMPS) and/or its partially or completely neutralized forms.

The sulphonated polymers according to the invention can be crosslinked or noncrosslinked. Preferably, crosslinked sulphonated polymers are chosen.

The term “partially neutralized” is understood to mean, within the meaning of the present invention, that sulpho groups in the salified form are present in the sulphonated polymer in a proportion of 1% to 99%.

Preferably, the sulphonated polymers in accordance with the invention are partially or completely neutralized with an inorganic base (sodium hydroxide, potassium hydroxide, ammonia) and/or an organic base, such as mono-, di- or triethanolamine, an aminomethylpropanediol, N-methylglucamine, basic amino acids, such as arginine and lysine, and mixtures of these compounds.

Advantageously, the sulphonated polymers are neutralized at least at 90%, that is to say that they comprise at least 90% of sulpho groups in the salified form.

Preferably, the sulphonated polymer or polymers is/are crosslinked poly(2-acrylamido-2-methylpropanesulphonic acid) polymers neutralized at least at 90%.

The crosslinked poly(2-acrylamido-2-methylpropanesulphonic acid) polymers neutralized at least at 90% comprise:

(a) from 90 to 99.9% by weight of units of following general formula (A):

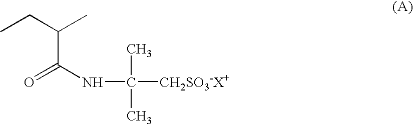

in which X⁺ is a proton, an alkali metal cation, an alkaline earth metal cation or the ammonium ion;

(b) from 0.01 to 10% by weight of crosslinking units originating from at least one monomer having at least two olefinic double bonds; the proportions by weight being defined with respect to the total weight of the polymer.

Preferably, the crosslinked poly(2-acrylamido-2-methylpropanesulphonic acid) polymers neutralized at least at 90% comprise from 98 to 99.5% by weight of units of formula (I) and from 0.2 to 2% by weight of crosslinking units.

More particularly, 90 to 100 mol % of the cations are NH₄⁺ cations and 0 to 10 mol % are protons.

The crosslinking monomers having at least two olefinic double bonds can be chosen, for example, from divinylbenzene, diallyl ether, dipropylene glycol diallyl ether, polyglycol diallyl ethers, triethylene glycol divinyl ether, hydroquinone diallyl ether, ethylene glycol di(meth)acrylate, tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl(meth)acrylate, allyl ethers of alcohols of the series of the sugars, or other allyl or vinyl ethers of polyfunctional alcohols, and allyl esters of phosphoric and/or vinylphosphonic acid derivatives, or mixtures of these compounds.

Use will more particularly be made of methylenebisacrylamide, allyl methacrylate or trimethylolpropane triacrylate (TMPTA).

The crosslinked poly(2-acrylamido-2-methylpropanesulphonic acid) polymers neutralized at least at 90% exhibit a viscosity, measured on a Brookfield viscosimeter, spindle 4, at a rotational speed of 100 revolutions/minute, in a 2% solution in water and at 25° C., of greater than or equal to 1000 cPs.

Preferably, the crosslinked poly(2-acrylamido-2-methylpropanesulphonic acid) polymers exhibit a viscosity, measured with a Brookfield viscosimeter, spindle 4, at a rotational speed of 100 revolutions/minute, in a 2% solution in water and at 25° C., ranging from 5000 to 40 000 cPs and more particularly from 6500 to 35 000 cPs.

The sulphonated polymers which can be used in the cosmetic composition in accordance with the invention can also be chosen from random amphiphilic AMPS polymers modified by reaction with a mono(C₆-C₂₂ n-alkyl)amine or a di(C₆-C₂₂ n-alkyl)amine, such as those described in Patent Application WO 00/31154, the content of which is incorporated in the present description.

These polymers can also additionally comprise other units derived from hydrophilic monomers comprising ethylenic unsaturation and not comprising a fatty chain.

These monomers comprising ethylenic unsaturation and not comprising a fatty chain can be chosen, for example, from (meth)acrylic acids, their alkyl derivatives substituted at the β position or their esters obtained with monoalcohols or mono- or polyalkylene glycols, (meth)acrylamides, vinylpyrrolidone, maleic anhydride, itaconic acid, maleic acid and mixtures of these compounds.

The sulphonated polymers can also be chosen from amphiphilic copolymers of AMPS and of at least one hydrophobic monomer comprising ethylenic unsaturation and comprising at least one hydrophobic part having from 6 to 50 carbon atoms, and more preferably from 6 to 22 carbon atoms, more preferably still from 6 to 18 carbon atoms, more particularly from 10 to 18 carbon atoms and more particularly still from 12 to 18 carbon atoms.

The same copolymers can additionally comprise one or more units derived from ethylenically unsaturated monomers not comprising a fatty chain, such as (meth)acrylic acids, their alkyl derivatives substituted at the β position or their esters obtained with monoalcohols or mono- or polyalkylene glycols, (meth)acrylamides, vinylpyrrolidone, maleic anhydride, itaconic acid or maleic acid, or the mixtures of these compounds.

These copolymers are described in particular in Patent Application EP-A-750 899 and U.S. Pat. No. 5,089,578 and in the following publications by Yotaro Morishima:

“Self-assembling amphiphilic polyelectrolytes and their nanostructures”, Chinese Journal of Polymer Science Vol. 18, No. 40 (2000), 323-336;

“Micelle formation of random copolymers of sodium 2-(acrylamido)-2-methylpropanesulfonate and a non-ionic surfactant macromonomer in water as studied by fluorescence and dynamic light scattering”, Macromolecules 2000, Vol. 33, No. 10, 3694-3704;

“Solution properties of micelle networks formed by non-ionic moieties covalently bound to an polyelectrolyte: salt effects on rheological behavior”, Langmuir, 2000, Vol. 16, No. 12, 5324-5332;

“Stimuli responsive amphiphilic copolymers of sodium 2-(acrylamido)-2-methylpropanesulfonate and associative macro-monomers”, Polym. Preprint, Div. Polym. Chem., 1999, 40(2), 220-221.

The hydrophobic monomers comprising ethylenic unsaturation of these specific copolymers are preferably chosen from acrylates or acrylamides of following formula (B):

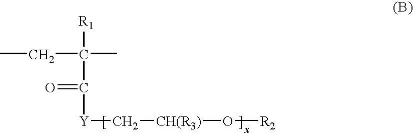

in which R₁ and R₃, which are identical or different, denote a hydrogen atom or a linear or branched C₁-C₆ alkyl radical (preferably methyl); Y denotes O or NH; R₂ denotes a hydrophobic hydrocarbon radical comprising from 6 to 50 carbon atoms, more preferably from 6 to 22 carbon atoms, or preferably still from 6 to 18 carbon atoms, more particularly from 10 to 18 carbon atoms and more particularly still from 12 to 18 carbon atoms; and x denotes a number of moles of alkylene oxide and varies from 0 to 100.

The R₂ radical is preferably chosen from linear C₆-C₁₈ alkyl radicals (for example, n-hexyl, n-octyl, n-decyl, n-hexadecyl or n-dodecyl) or branched or cyclic C₆-C₁₈ alkyl radicals (for example, cyclododecane (C₁₂) or adamantane (C₁₀)); perfluorinated C₆-C₁₈ alkyl radicals (for example, the group of formula —(CH₂)₂—(CF₂)₉—CF₃); the cholesteryl radical (C₂₇) or a cholesterol ester residue, such as the cholesteryl oxyhexanoate group; or polycyclic aromatic groups, such as naphthalene or pyrene. Preference is more particularly given, among these radicals, to linear alkyl radicals and more particularly the n-dodecyl radical.

According to a particularly preferred form of the invention, the monomer of formula (B) comprises at least one alkylene oxide unit (x≧2) and preferably a polyoxyalkylene chain. The polyoxyalkylene chain is preferably composed of ethylene oxide units and/or propylene oxide units and more particularly still is composed of ethylene oxide units. The number of oxyalkylene units generally varies from 3 to 100, more preferably from 3 to 50 and more preferably still from 7 to 25.

Mention may be made, among these polymers, of:
crosslinked or noncrosslinked and neutralized or non-neutralized copolymers comprising from 15 to 60% by weight of AMPS units and from 40 to 85% by weight of (C₈-C₁₆)alkyl(meth)acrylamide units or of (C₈-C₁₆)alkyl(meth)acrylate units with respect to the polymer, such as those described in Application EP-A-750 899;
terpolymers comprising from 10 to 90 mol % of acrylamide units, from 0.1 to 10 mol % of AMPS units and from 5 to 80 mol % of n-(C₆-C₁₈)alkylacrylamide units, such as those described in U.S. Pat. No. 5,089,578.

Mention may also be made of noncrosslinked copolymers of partially or completely neutralized AMPS and of dodecyl methacrylate, and noncrosslinked or crosslinked copolymers of partially or completely neutralized AMPS and of n-dodecyl-methacrylamide, such as those described in the above-mentioned papers by Morishima.

Mention will more particularly be made of the copolymers composed of 2-acrylamido-2-methylpropanesulphonic acid (AMPS) units of following formula (A):

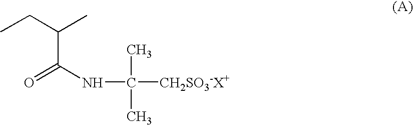

in which X⁺ is a proton, an alkali metal cation, an alkaline earth metal cation or the ammonium ion,

and of units of following formula (C):

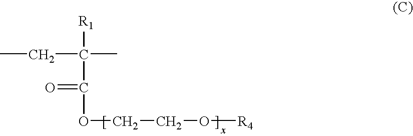

in which x denotes an integer varying from 3 to 100, preferably from 5 to 80 and more preferably from 7 to 25; R₁ has the same meaning as that indicated above in the formula (A) and R₄ denotes a linear or branched C₆-C₂₂ and more preferably C₁₀-C₂₂ alkyl.

The particularly preferred polymers are those for which x=25, R₁ denotes methyl and R₄ represents n-dodecyl; they are described in the abovementioned papers by Morishima.

The polymers for which X⁺ denotes sodium or ammonium are more particularly preferred.

The molar concentration in % of the units of formula (A) and of the units of formula (C) in the sulphonated polymers which can be used in the cosmetic composition according to the invention can vary between 0.1 and 99.9 mol %.

Preferably, for the most hydrophobic polymers, the molar proportion of units of formula (B) or (C) varies from 50.1 to 99.9%, more particularly from 70 to 95% and more particularly still from 80 to 90%.

Preferably, for the polymers which are not very hydrophobic, the molar proportion of units of formula (B) or (C) varies from 0.1 to 50%, more particularly from 5 to 25% and more particularly still from 10 to 20%.

The sulphonated polymers which can be used in the cosmetic composition in accordance with the invention can be obtained according to conventional radical polymerization processes in the presence of one or more initiators, such as, for example, azobisisobutyronitrile (AIBN), azobisdimethylvaleronitrile, ABAH (2,2-azobis[2-amidinopropane]hydrochloride), organic peroxides, such as dilauryl peroxide, benzoyl peroxide, tert-butyl hydroperoxide and the like, inorganic peroxide compounds, such as potassium or ammonium persulphate, or H₂O₂, optionally in the presence of reducing agents.

They are in particular obtained by radical polymerization in a tert-butanol medium from which they precipitate.

Furthermore, by using polymerization by precipitation from tert-butanol, it is possible to obtain a size distribution of the sulphonated polymer particles which is particularly favourable for its uses.

The size distribution of the polymer particles can be determined, for example, by laser scattering or image analysis.

An advantageous distribution for this type of sulphonated polymer determined by image analysis is as follows: 60.2% less than 423 microns, 52.0% less than 212 microns, 26.6% less than 106 microns, 2.6% less than 45 microns and 26.6% greater than 850 microns.

The reaction can be carried out at a temperature of between 10 and 150° C., either at atmospheric pressure or under reduced pressure. It can also be carried out under an inert atmosphere and preferably under nitrogen.

For example, poly(2-acrylamido-2-methylpropanesulphonic acid)s can be obtained according to a preparation process comprising the following stages:

(a) the 2-acrylamide-2-methylpropanesulphonic acid monomer is dispersed or dissolved in the free form in a solution of tert-butanol or of water and of tert-butanol;

(b) the solution or the dispersion of 2-acrylamido-2-methylpropanesulphonic acid monomer obtained during stage (a) is neutralized with one or more inorganic or organic bases, preferably ammonia NH₃, in an amount which makes it possible to obtain a degree of neutralization of the sulphonic acid functional groups of the polymer ranging from 90 to 100%;

(c) the crosslinking monomer or monomers is/are added to the solution or dispersion in (b);

(d) a conventional radical polymerization is carried out in the presence of free radical initiators at a temperature ranging from 10 to 150° C.; the polymer precipitating from the solution or dispersion based on tert-butanol.

The composition according to the invention preferably comprises at least 0.01% by weight of sulphonated polymer(s), with respect to the total weight of the composition.

More preferably, the composition according to the invention comprises from 0.01% to 10% by weight of sulphonated polymer(s), more preferably still from 0.05% to 5% by weight and better still from 0.1% to 3% by weight, with respect to the total weight of the composition.

The ratio by weight of the amount of copolymer(s) of acrylate(s) or of methacrylate(s) and of hydroxyester of acrylate(s) or of methacrylate(s), on the one hand, to the amount of sulphonated polymer(s), on the other hand, is between 0.1 and 10 and preferably between 1 and 8.

The compositions according to the invention can also comprise at least one fatty substance. The fatty substances which can be used in the present invention are chosen in particular from plant oils, animal oils, mineral oils, natural or synthetic oils, fatty alcohols, waxes and their mixtures.

Mention may in particular be made, as plant oils, of sweet almond oil, avocado oil, castor oil, olive oil, liquid jojoba wax, sunflower oil, wheat germ oil, sesame oil, groundnut oil, grape seed oil, soybean oil, rapeseed oil, safflower oil, coconut oil, maize oil, hazelnut oil, palm oil, apricot kernel oil, calophyllum oil, evening primrose oil, shea butter, rice bran oil, maize germ oil, passionflower oil and rye oil.

Mention may in particular be made, as animal oil, of perhydrosqualene.

Mention may in particular be made, as mineral oil, of liquid paraffin and liquid petrolatum.

Mention may in particular be made, as synthetic oil, of squalane, poly(α-olefin)ₛ, such as isododecane or isohexadecane, transesterified vegetable oils, fluorinated oils or fatty esters.

The term “fatty acids” denotes compounds of formula RₐCOOR_{b} in which Rₐ represents the residue of a saturated or unsaturated, hydroxylated or nonhydroxylated and linear or branched higher acid comprising from 4 to 29 carbon atoms and R_{b} represents a saturated or unsaturated and linear or branched hydrocarbon chain comprising from 3 to 30 carbon atoms, the total number of carbon atoms of the ester being greater than 10. Mention may in particular be made, by way of examples, of Purcellin oil (stearyl octanoate), isopropyl myristate, isopropyl palmitate, butyl stearate, hexyl laurate, isononyl isononanoate, 2-ethylhexyl palmitate, 2-hexyldecyl laurate, 2-octyldecyl palmitate, 2-octyldodecyl myristate, isostearyl neopentanoate or tridecyl neopentanoate.

The preferred fatty alcohols comprise myristyl alcohol, cetyl alcohol, stearyl alcohol, arachidyl alcohol, behenyl alcohol and erucyl alcohol.

A wax within the meaning of the present invention is a lipophilic compound which is solid at ambient temperature (approximately 25° C.) with a reversible solid/liquid change in state, which has a melting point of greater than approximately 40° C. which can range up to 200° C., and which exhibits, in the solid state, an anisotropic crystalline arrangement. Animal and vegetable waxes comprise, as essential constituents, esters of carboxylic acids and of alcohols comprising long chains. Generally, the size of the crystals of the wax is such that the crystals diffract and/or scatter light, conferring a more or less opaque cloudy appearance on the composition comprising them. By bringing the wax to its melting point, it is possible to render it miscible with oils and to form a microscopically homogeneous mixture but, on returning the temperature to ambient temperature, recrystallization of the wax in the oils of the mixture, detectable microscopically and macroscopically (opalescence), is obtained.

Mention may be made, as waxes which can be used in the present invention, of waxes of animal origin, such as beeswax, spermaceti, lanoline wax and lanoline derivatives; vegetable waxes, such as sunflower wax, rice wax, apple wax, carnauba wax, candelilla wax, ouricury wax, japon wax, cocoa butter or cork fibre or sugarcane waxes; mineral waxes, for example paraffin wax, petrolatum wax, lignite wax, microcrystalline waxes, ceresin or ozokerite; synthetic waxes, such as polyethylene waxes or Fischer-Tropsch waxes, and their mixtures.

The fatty substances as described above, when they are present in the composition according to the invention, are preferably present in an amount ranging from 0.1 to 30% by weight, preferably from 1 to 20% by weight and better still from 5 to 15% by weight, with respect to the total weight of the composition.

The composition according to the invention can also comprise at least one surfactant chosen from cationic surfactants, anionic surfactants, non-ionic surfactants, or amphoteric or zwitterionic surfactants.

The composition according to the invention then preferably comprises at least 0.01% by weight of surfactant(s), with respect to the total weight of the composition. Preferably, the composition according to the invention comprises from 0.01 to 20% by weight of surfactant(s), more preferably from 0.05 to 4% by weight, with respect to the total weight of the composition.

Mention may in particular be made, as example of anionic surfactants which can be used in the compositions according to the present invention, of (nonlimiting list) the salts (in particular alkali metal salts, especially sodium salts, ammonium salts, amine salts, aminoalcohol salts or alkaline earth metal salts (magnesium salts)) of the following compounds: alkyl sulphates, alkyl ether sulphates, alkylamido ether sulphates, alkylaryl polyether sulphates, monoglyceride sulphates; alkylsulphonates, alkyl phosphates, alkylamidesulphonates, alkylarylsulphonates, α-olefinsulphonates, paraffinsulphonates; alkyl sulphosuccinates; alkyl ether sulphosuccinates, alkylamide sulphosuccinates; alkyl sulphosuccinamates; alkyl sulphoacetates; alkyl ether phosphates, acylsarcosinates; acylisethionates and N-acyltaurates, the alkyl or acyl radical of all of these various compounds preferably comprising from 12 to 20 carbon atoms and the aryl radical preferably denoting a phenyl or benzyl group.

Mention may also be made, among anionic surfactants which can also be used, of salts of fatty acids, such as salts of oleic acid, ricinoleic acid, palmitic acid or stearic acid; coconut oil acid or hydrogenated coconut oil acid; acyl lactylates, the acyl radical of which comprises from 8 to 20 carbon atoms.

Use may also be made of weakly anionic surfactants, such as alkyl-D-galactosideuronic acids and their salts, and also polyoxyalkylenated (C₆-C₂₄)alkyl ether carboxylic acids, polyoxyalkylenated (C₆-C₂₄)alkylaryl ether carboxylic acids, polyoxyalkylenated (C₆-C₂₄)alkylamido ether carboxylic acids and their salts, in particular those comprising from 2 to 50 ethylene oxide groups, and their mixtures.

Preference is given, among anionic surfactants, to the use according to the invention of alkyl sulphate and alkyl ether sulphate salts and their mixtures.

The cationic surfactants which can be used in the compositions of the present invention comprise, for example, salts of optionally polyoxyalkylenated primary, secondary or tertiary fatty amines, quaternary ammonium salts and their mixtures.

Mention may in particular be made, as quaternary ammonium salts, of, for example:
those corresponding to the following general formula (I):

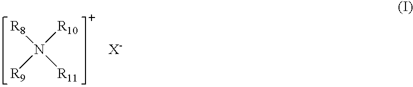

in which the radicals R₈ to R₁₁, which can be identical or different, represent a linear or branched aliphatic radical comprising from 1 to 30 carbon atoms or an aromatic radical, such as aryl or alkylaryl. The aliphatic radicals can comprise heteroatoms, such as in particular oxygen, nitrogen, sulphur and halogens. The aliphatic radicals are, for example, chosen from C₁₋₃₀ alkyl, C₁₋₃₀ alkoxy, polyoxy(C₂-C₆) alkylene, C₁₋₃₀ alkylamide, (C₁₂-C₂₂)alkylamino(C₂-C₆)alkyl, (C₁₂-C₂₂)alkyl acetate and C₁₋₃₀ hydroalkyl radicals; X is an anion chosen from the group of the halides, phosphates, acetates, lactates, (C₂-C₆)alkyl sulphates, or alkyl- or arylsulphonates.

Preference is given, among quaternary ammonium salts of formula (I), on the one hand, to tetraalkylammonium chlorides, such as, for example, dialkyldimethylammonium or alkyltrimethylammonium chlorides in which the alkyl radical comprises approximately from 12 to 22 carbon atoms, in particular behenyltrimethylammonium chloride, distearyldimethylammonium chloride, cetyltrimethylammonium chloride or benzyldimethylstearylammonium chloride, or also, on the other hand, to palmitylamidopropyltrimethylammonium chloride or stearamidopropyldimethyl(myristyl acetate)ammonium chloride, sold under the name Ceraphyl® 70 by Van Dyk.
imidazoline quaternary ammonium salts, such as, for example, those of following formula (II):

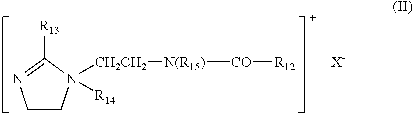

in which R₁₂ represents an alkenyl or alkyl radical comprising from 8 to 30 carbon atoms, for example derivatives of tallow fatty acids, R₁₃ represents a hydrogen atom, a C₁-C₄ alkyl radical or an alkenyl or alkyl radical comprising from 8 to 30 carbon atoms, R₁₄ represents a C₁-C₄ alkyl radical, R₁₅ represents a hydrogen atom or a C₁-C₄ alkyl radical, and X⁻ is an anion chosen from the group of the halides, phosphates, acetates, lactates, alkylsulphates, or alkyl- or alkylarylsulphonates. Preferably, R₁₂ and R₁₃ denote a mixture of alkenyl or alkyl radicals comprising from 12 to 21 carbon atoms, for example derivatives of tallow fatty acids, R₁₄ denotes a methyl radical, and R₁₅ denotes a hydrogen atom. Such a product is sold, for example, under the name Rewoquat® W 75 by Rewo;
diquaternary ammonium salts of formula (III):

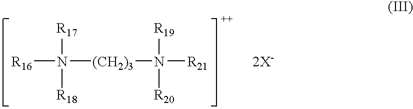

in which R₁₆ denotes an aliphatic radical comprising approximately from 16 to 30 carbon atoms, R₁₇, R₁₈, R₁₉, R₂₀ and R₂₁, which are identical or different, are chosen from a hydrogen atom and an alkyl radical comprising from 1 to 4 carbon atoms, and X is an anion chosen from the group of the halides, acetates, phosphates, nitrates and methyl sulphates. Such diquaternary ammonium salts comprise in particular propanetallowediammonium dichloride;
quaternary ammonium salts comprising at least one ester functional group, such as those of following formula (IV):

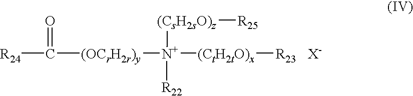

in which:
R₂₂ is chosen from C₁-C₆ alkyl radicals and C₁-C₆ hydroxylalkyl or dihydroxyalkyl radicals;
R₂₃ is chosen from:
the

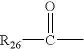

radical
saturated or unsaturated and linear or branched C₁-C₂₂ hydrocarbon radicals R₂₇.
the hydrogen atom,
   R₂₅ is chosen from:
the

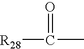

radical
saturated or unsaturated and linear or branched C₁-C₆ hydrocarbon radicals R₂₉,
the hydrogen atom,
   R₂₄, R₂₆ and R₂₈, which are identical or different, are chosen from saturated or unsaturated and linear or branched C₇-C₂, hydrocarbon radical;
   r, s and t, which are identical or different, are integers having values from 2 to 6;
   y is an integer having a value from 1 to 10;
   x and z, which are identical or different, are integers having values from 0 to 10;
   X⁻ is an organic or inorganic and simple or complex anion;
   with the proviso that the sum x+y+z has a value from 1 to 15, that, when x has a value of 0, then R₂₃ denotes R₂₇ and that, when z has a value of 0, then R₂₅ denotes R₂₉.

The R₂₂ alkyl radicals can be linear or branched and more particularly linear.

Preferably, R₂₂ denotes a methyl, ethyl, hydroxyethyl or dihydroxypropyl radical and more particularly a methyl or ethyl radical.

Advantageously, the sum x+y+z has a value from 1 to 10.

When R₂₃ is an R₂₇ hydrocarbon radical, it can be long and have from 12 to 22 carbon atoms or short and have from 1 to 3 carbon atoms.

When R₂₅ is an R₂₉ hydrocarbon radical, it preferably has from 1 to 3 carbon atoms.

Advantageously, R₂₄, R₂₆ and R₂₈, which are identical or different, are chosen from saturated or unsaturated and linear or branched C₁₁-C₂₁ hydrocarbon radicals and more particularly from saturated or unsaturated and linear or branched C₁₁-C₂₁ alkyl and alkenyl radicals.

Preferably, x and z, which are identical or different, have values of 0 or 1.

Advantageously, y is equal to 1.

Preferably, r, s and t, which are identical or different, have values of 2 or 3 and more particularly still are equal to 2.

The anion is preferably a halide (chloride, bromide or iodide) or an alkyl sulphate, more particularly methyl sulphate. However, use may be made of methanesulphonate, phosphate, nitrate, tosylate, an anion derived from an organic acid, such as acetate or lactate, or any other anion compatible with the ammonium comprising an ester functional group.

The anion X⁻ is more particularly still chloride or methyl sulphate.

Use is more particularly made, in the composition according to the invention, of the ammonium salts of formula (IV) in which:
R₂₂ denotes a methyl or ethyl radical,
x and y are equal to 1;
z is equal to 0 or 1;
r, s and t are equal to 2;
R₂₃ is chosen from:
   the

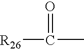

radical
methyl, ethyl or C₁₄-C₂₂ hydrocarbon radicals,
the hydrogen atom;
R₂₅ is chosen from:
   the

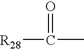

radical
the hydrogen atom;
R₂₄, R₂₆ and R₂₈, which are identical or different, are chosen from saturated or unsaturated and linear or branched C₁₃-C₁₇ hydrocarbon radicals and preferably from saturated or unsaturated and linear or branched C₁₃-C₁₇ alkyl and alkenyl radicals.

Advantageously, the hydrocarbon radicals are linear.

Mention may be made, for example, of compounds of formula (IV), such as diacyloxyethyldimethylammonium, diacyloxyethyl(hydroxyethyl)methylammonium, monoacyloxyethyldi(hydroxyethyl)methylammonium, triacyloxyethylmethylammonium or monoacyloxyethyl(hydroxyethyl)dimethylammonium salts (in particular chloride or methyl sulphate), and their mixtures. The acyl radicals preferably have from 14 to 18 carbon atoms and originate more particularly from a vegetable oil, such as palm oil or sunflower oil. When the compound comprises several acyl radicals, the latter can be identical or different.

These products are obtained, for example, by direct esterification of triethanolamine, triisopropanolamine, alkyldiethanolamine or alkyldiisopropanolamine, optionally oxyalkylenated, with fatty acids or with mixtures of fatty acids of vegetable or animal origin, or by transesterification of their methyl esters. This esterification is followed by quaternization using an alkylating agent, such as an alkyl halide (preferably methyl or ethyl halide), a dialkyl sulphate (preferably dimethyl or diethyl sulphate), methyl methanesulphonate, methyl para-toluenesulphonate, glycol chlorohydrin or glycerol chlorohydrin.

Such compounds are sold, for example, under the names Dehyquart® by Henkel, Stepanquat® by Stepan, Noxamium® by Ceca or Rewoquat® WE 18 by Rewo-Witco.

The composition according to the invention can, for example, comprise a mixture of quaternary ammonium mono-, di- and triester salts with a predominance by weight of diester salts.

The use may be made, as mixture of ammonium salts, for example, of the mixture comprising from 15 to 30% by weight of acyloxyethyldi(hydroxyethyl)methylammonium methyl sulphate from 45 to 60% by weight of diacyloxyethyl(hydroxyethyl)methylammonium methyl sulphate and from 15 to 30% by weight of triacyloxyethylmethylammonium methyl sulphate, the acyl radicals having from 14 to 18 carbon atoms and originating from palm oil which is optionally partially hydrogenated.

Use may also be made of the ammonium salts comprising at least one ester functional group described in U.S. Pat. No. 4,874,554 and U.S. Pat. No. 4,137,180.

The non-ionic surfactants which can be used in the compositions of the present invention are compounds well known per se (see in particular in this respect “Handbook of Surfactants” by M. R. Porter, published by Blackie & Son (Glasgow and London), 1991, pp. 116-178). They are chosen in particular from polyethoxylated, polypropoxylated or polyglycerolated alcohols and fatty alcohols, polyethoxylated, polypropoxylated or polyglycerolated α-diols, polyethoxylated, polypropoxylated or polyglycerolated (C₁₋₂₀)alkylphenols or polyethoxylated, polypropoxylated or polyglycerolated fatty acids, the fatty chain comprising, for example, from 8 to 18 carbon atoms, it being possible for the number of ethylene oxide or propylene oxide groups to range in particular from 2 to 50 and it being possible for the number of glycerol groups to range in particular from 2 to 30.

Mention may also be made of condensates of ethylene oxide and of propylene oxide with fatty alcohols; polyethoxylated fatty amides preferably having from 2 to 30 ethylene oxide units, polyglycerolated fatty amides comprising on average from 1 to 5 glycerol groups and in particular from 1.5 to 4, ethoxylated esters of fatty acids and of sorbitan having from 2 to 30 ethylene oxide units, sucrose fatty acid esters, esters of fatty acids and of polyethylene glycol, alkylpolyglycosides, polyethoxylated vegetable oils, N—(C₆₋₂₄ alkyl)glucamine derivatives or amine oxides, such as (C₁₀₋₁₄ alkyl)amine oxides or N—(C₁₀₋₁₄ acyl)aminopropylmorpholine oxides.

The amphoteric or zwitterionic surfactants which can be used in the compositions of the present invention comprise, for example, aliphatic secondary or tertiary amine derivatives in which the aliphatic group is a linear or branched chain comprising from 8 to 22 carbon atoms and comprising at least one anionic group, such as, for example, a carboxylate, sulphonate, sulphate, phosphate or phosphonate group. Mention may also be made of (C₈₋₂₀)alkyl betaines, sulphobetaines, (C₈₋₂₀ alkyl) amido(C₆₋₈ alkyl) betaines or (C₈₋₂₀ alkyl) amido(C₆₋₈ alkyl) sulphobetaines.

Mention may be made, among amine derivatives, of the products sold under the name Miranol®, such as described in U.S. Pat. No. 2,528,378 and U.S. Pat. No. 2,781,354 and classified in the CTFA dictionary, 3rd Edition, 1982, under the names Amphocarboxyglycinate and Amphocarboxypropionate with the respective structures (1) and (2):

Rₐ—CONHCH₂CH₂—N(R_{b})(R_{c})(CH₂COO—) (1)

in which:

Rₐ represents an alkyl group derived from an acid Rₐ—COOH present in hydrolysed coconut oil or a heptyl, nonyl or undecyl group,

R_{b} represents a β-hydroxyethyl group, and

R_{c} represents a carboxymethyl group; and

Rₐ′—CONHCH₂CH₂—N(B)(B′) (2)

in which:

B represents —CH₂CH₂OX′,

B′ represents —(CH₂)_{z}—Y′, with z=1 or 2,

X′ represents the —CH₂CH₂—COOH group or a hydrogen atom,

Y′ represents —COOH or the —CH₂—CHOH—SO₃H group,

Rₐ′ represents an alkyl group of an acid Rₐ′—COOH present in hydrolysed coconut oil or in hydrolysed linseed oil, an alkyl group, in particular a C₁₇ alkyl group and its iso form, or an unsaturated C₁₇ group.

These compounds are classified in the CTFA dictionary, 5th Edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

Mention may be made, by way of example, of the cocoamphodiacetate sold by Rhodia under the trade name Miranol® C2M Concentrate.

The compositions according to the invention can also additionally comprise at least one silicone or one silicone derivative in the soluble, dispersed or microdispersed form. The silicones or silicone derivatives are preferably present in an amount of less than 10% by weight, preferably ranging from 0.01 to 8% by weight and more preferably still from 0.1 to 5% by weight, with respect to the total weight of the composition.

Mention may in particular be made, by way of example, of silicone oils, such as, for example, linear or cyclic polydimethylsiloxanes.

The compositions according to the invention can also comprise at least one additional fixing polymer, other than the copolymer of acrylate(s) or of methacrylate(s) and of hydroxyester of acrylate(s) or of methacrylate(s), in order to strengthen the form retention effect. Such an additional fixing polymer can be anionic, cationic, non-ionic or amphoteric in nature and preferably anionic, non-ionic or amphoteric in nature.

The anionic fixing polymers which can be used in the compositions according to the invention are polymers comprising groups derived from carboxylic acid which have a number-average molecular weight of between approximately 500 and 5 000 000.

The carboxyl groups are contributed by unsaturated mono- or dicarboxylic acid monomers such as those corresponding to the formula:

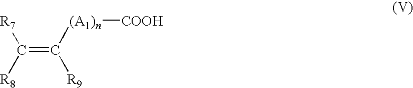

in which n is an integer from 0 to 10, A₁ denotes a methylene group, optionally connected to the carbon atom of the unsaturated group or to the neighbouring methylene group when n is greater than 1 via a heteroatom, such as oxygen or sulphur, R₇ denotes a hydrogen atom or a phenyl or benzyl group, R₈ denotes a hydrogen atom or a lower alkyl or carboxyl group, and R₉ denotes a hydrogen atom, a lower alkyl group or a —CH₂—COOH, phenyl or benzyl group.

In the abovementioned formula, a lower alkyl group preferably denotes a group having 1 to 4 carbon atoms and in particular methyl and ethyl groups.

The preferred anionic fixing polymers possessing carboxyl groups according to the invention are:

A) Copolymers of acrylic acid and of acrylamide.

B) Copolymers of acrylic or methacrylic acid with a monoethylenic monomer chosen from ethylene, styrene, vinyl esters or esters of acrylic or methacrylic acid, optionally grafted onto a polyalkylene glycol, such as polyethylene glycol, and optionally crosslinked. Such polymers are described in particular in French Patent No. 1 222 944 and German Application No. 2 330 956, the copolymers of this type comprising, in their chain, an optionally N-alkylated and/or -hydroxyalkylated acrylamide unit, such as described in particular in Luxembourgian Patent Applications Nos. 75370 and 75371. Mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as that sold by ISP under the name Acrylidone® LM, and methacrylic acid/ethyl acrylate/tert-butyl acrylate terpolymers, such as the product sold under the name Luvimer® 100 P by BASF.

C) Copolymers derived from crotonic acid, such as those comprising, in their chain, vinyl acetate or propionate units and optionally other monomers, such as allyl or methallyl esters, vinyl ether or vinyl ester of a linear or branched saturated carboxylic acid comprising a long hydrocarbon chain, such as those comprising at least 5 carbon atoms, it optionally being possible for these polymers to be grafted and crosslinked, or alternatively another monomer which is a vinyl, allyl or methallyl ester of an α- or β-cyclic carboxylic acid. Such polymers are described, inter alia, in French Patents Nos. 1 222 944, 1 580 545, 2 265 782, 2 265 781, 1 564 110 and 2 439 798. A commercial product coming within this class is Resin 28-29-30, sold by National Starch.

D) Copolymers derived from C₄-C₈ monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, or acrylic acid and its esters, the anhydride functional groups of these copolymers optionally being monoesterified or monoamidated. Such polymers are described in particular in U.S. Pat. Nos. 2,047,398, 2,723,248 and 2,102,113 and GB Patent No. 839 805. Commercial products are in particular those sold under the names Gantrez® AN or ES by ISP;
copolymers comprising (i) one or more maleic, citraconic or itaconic anhydride units and (ii) one or more monomers chosen from allyl or methallyl esters, optionally comprising one or more acrylamide, methacrylamide, α-olefin, acrylic or methacrylic ester, acrylic or methacrylic acid, or vinylpyrrolidone groups in their chain,

the anhydride functional groups of these copolymers optionally being monoesterified or monoamidated.

These polymers are, for example, described in French Patents Nos. 2 350 384 and 2 357 241 of the Applicant Company.

E) Polyacrylamides comprising carboxylate groups.

According to the invention, preference will be given, among the anionic fixing polymers mentioned above, to acrylic acid copolymers, such as the acrylic acid/ethyl acrylate/N-tert-butylacrylamide terpolymers sold in particular under the name Ultrahold® Strong by BASF, copolymers derived from crotonic acid, such as the vinyl acetate/vinyl tert-butylbenzoate/crotonic acid terpolymers and the crotonic acid/vinyl acetate/vinyl neododecanoate terpolymers sold in particular under the name Resin 28-29-30 by National Starch, polymers derived from maleic, fumaric or itaconic acids or anhydrides with vinyl esters, vinyl ethers, vinyl halides, phenylvinyl derivatives, or acrylic acid and its esters, such as the monoesterified methyl vinyl ether/maleic anhydride copolymers sold, for example, under the name Gantrez® by ISP, the copolymers of methacrylic acid and of methyl methacrylate sold under the name Eudragit® L by Röhm Pharma, the copolymers of methacrylic acid and of ethyl acrylate sold under the name Luvimer® MAEX or MAE by BASF, vinyl acetate/crotonic acid copolymers and the vinyl acetate/crotonic acid copolymers grafted with polyethylene glycol sold under the name Aristoflex® A by BASF.

The most particularly preferred anionic fixing polymers mentioned above are chosen from the monoesterified methyl vinyl ether/maleic anhydride copolymers sold under the name Gantrez® ES 425 by ISP, the acrylic acid/ethyl acrylate/N-tert-butylacrylamide terpolymers sold under the name Ultrahold® Strong by BASF, the copolymers of methacrylic acid and of methyl methacrylate sold under the name Eudragit® L by Röhm Pharma, the vinyl acetate/vinyl tert-butylbenzoate/crotonic acid terpolymers and the crotonic acid/vinyl acetate/vinyl neododecanoate terpolymers sold under the name Resin 28-29-30 by National Starch, the copolymers of methacrylic acid and of ethyl acrylate sold under the name Luvimer® MAEX or MAE by BASF or the vinylpyrrolidone/acrylic acid/lauryl methacrylate terpolymers sold under the name Acrylidone® LM by ISP.

The amphoteric fixing polymers which can be used in accordance with the invention can be chosen from polymers comprising B and C units distributed randomly in the polymer chain, where B denotes a unit deriving from a monomer comprising at least one basic nitrogen atom and C denotes a unit deriving from an acidic monomer comprising one or more carboxyl or sulpho groups or else B and C can denote groups deriving from zwitterionic carboxybetaine or sulphobetaine monomers;

B and C can also denote a cationic polymer chain comprising primary, secondary, tertiary or quaternary amine groups, in which at least one of the amine groups carries a carboxyl or sulpho group connected via a hydrocarbon group, or else B and C form part of a chain of a polymer comprising an α,β-dicarboxyethylene unit, one of the carboxyl groups of which has been reacted with a polyamine comprising one or more primary or secondary amine groups.

The more particularly preferred amphoteric fixing polymers corresponding to the definition given above are chosen from the following polymers:

1) Polymers resulting from the copolymerization of a monomer derived from a vinyl compound carrying a carboxyl group, such as more particularly acrylic acid, methacrylic acid, maleic acid or α-chloroacrylic acid, and of a basic monomer derived from a substituted vinyl compound comprising at least one basic atom, such as more particularly dialkylaminoalkyl methacrylate and acrylate or dialkylaminoalkylmethacrylamide and -acrylamide. Such compounds are described in U.S. Pat. No. 3,836,537.

2) Polymers comprising units deriving:
a) from at least one monomer chosen from acrylamides or methacrylamides substituted on the nitrogen atom with an alkyl group,
b) from at least one acidic comonomer comprising one or more reactive carboxyl groups, and
c) from at least one basic comonomer, such as esters possessing primary, secondary, tertiary and quaternary amine substituents of acrylic and methacrylic acids and the quaternization product of dimethylaminoethyl methacrylate with dimethyl or diethyl sulphate.

The more particularly preferred N-substituted acrylamides or methacrylamides according to the invention are the compounds in which the alkyl groups comprise from 2 to 12 carbon atoms and more particularly N-ethylacrylamide, N-tert-butylacrylamide, N-tert-octylacrylamide, N-octylacrylamide, N-decylacrylamide or N-dodecylacrylamide, and the corresponding methacrylamides.

The acidic comonomers are more particularly chosen from acrylic, methacrylic, crotonic, itaconic, maleic or fumaric acids and alkyl monoesters having 1 to 4 carbon atoms of maleic or fumaric acids or anhydrides.

The preferred basic comonomers are aminoethyl, butylaminoethyl, N,N′-dimethylaminoethyl or N-tert-butylaminoethyl methacrylates.

Use is particularly made of the copolymers for which the CTFA name (4th Ed., 1991) is Octylacrylamide/acrylates/butylaminoethyl methacrylate copolymer, such as the products sold under the name Amphomer® or Lovocryl® 47 by National Starch.

(3) Partially or completely acylated and crosslinked polyaminoamides deriving from polyaminoamides of general formula:

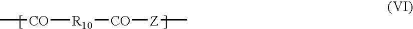

in which R₁₀ represents a divalent group derived from a saturated dicarboxylic acid, from an aliphatic mono- or dicarboxylic acid comprising an ethylenic double bond, from an ester of a lower alkanol having 1 to 6 carbon atoms of these acids, or from a group deriving from the addition of any one of the said acids with a bisprimary or bissecondary amine, and Z denotes a group deriving from a bisprimary, mono- or bissecondary polyalkylenepolyamine and preferably represents:

a) in the proportions of 60 to 100 mol %, the group

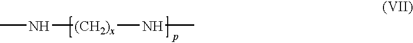

where x=2 and p=2 or 3, or else x=3 and p=2

this group deriving from diethylenetriamine, triethylenetetraamine or dipropylenetriamine;

b) in the proportions of 0 to 40 mol %, the above group (VIIa), in which x=2 and p=1 and which derives from ethylenediamine, or the group deriving from piperazine:

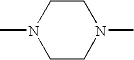

c) in the proportions of 0 to 20 mol %, the group —NH—(CH₂)₆—NH— deriving from hexamethylenediamine, these polyaminoamides being crosslinked by addition reaction of a bifunctional crosslinking agent chosen from epihalohydrins, diepoxides, dianhydrides or bisunsaturated derivatives, by means of 0.025 to 0.35 mol of crosslinking agent per amine group of the polyaminoamide, and acylated by reaction with acrylic acid, chloroacetic acid or an alkanesultone or their salts.

The saturated carboxylic acids are preferably chosen from acids having 6 to 10 carbon atoms, such as adipic, 2,2,4-trimethyladipic or 2,4,4-trimethyladipic, or terephthalic acids, and the acids comprising an ethylenic double bond, such as, for example, acrylic, methacrylic or itaconic acids.

The alkanesultones used in the acylation are preferably propane- or butanesultone and the salts of the acylating agents are preferably the sodium or potassium salts.

(4) Polymers comprising zwitterionic units of formula:

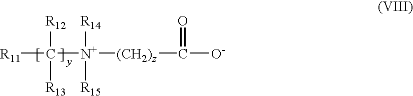

in which R₁₁ denotes a polymerizable unsaturated group, such as an acrylate, methacrylate, acrylamide or methacrylamide group, y and z represent an integer from 1 to 3, R₁₂ and R₁₃ represent a hydrogen atom or a methyl, ethyl or propyl group, and R₁₄ and R₁₅ represent a hydrogen atom or an alkyl group such that the sum of the carbon atoms in R₁₄ and R₁₅ does not exceed 10.

The polymers comprising such units can also comprise units derived from non-zwitterionic monomers, such as dimethyl- or diethylaminoethyl acrylate or methacrylate or alkyl acrylates or methacrylates, acrylamides or methacrylamides, or vinyl acetate.

Mention may be made, by way of example, of methyl methacrylate/methyl dimethylcarboxymethylammonioethyl methacrylate copolymers.

(5) Polymers derived from chitosan comprising monomer units corresponding to the following formulae:

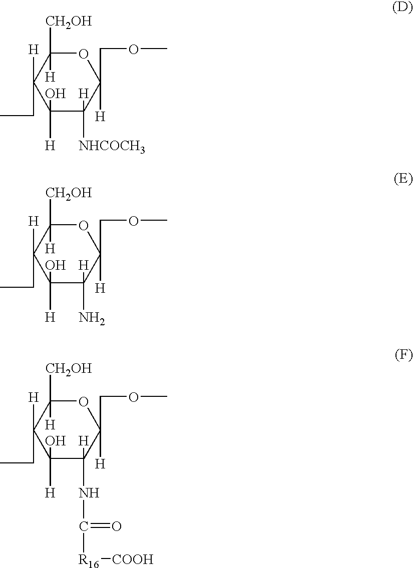

the unit (D) being present in proportions of between 0 and 30%, the unit (E) in proportions of between 5 and 50% and the unit (F) in proportions of between 30 and 90%, it being understood that, in this unit (F), R₁₆ represents a group of formula:

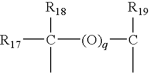

in which, if q=0, R₁₇, R₁₈ and R₁₉, which may be identical or different, each represent a hydrogen atom, a methyl, hydroxyl, acetoxy or amino residue, a monoalkylamino residue or a dialkylamino residue, optionally interrupted by one or more nitrogen atoms and/or optionally substituted by one or more amino, hydroxyl, carboxyl, alkylthio or sulpho groups, or an alkylthio residue in which the alkyl group carries an amino residue, at least one of the R₁₇, R₁₈ and R₁₉ groups being, in this case, a hydrogen atom;

or, if q=1, R₁₇, R₁₈ and R₁₉ each represent a hydrogen atom, and the salts formed by these compounds with bases or acids.

(6) Polymers corresponding to the general formula (IX), for example described in French Patent 1 400 366:

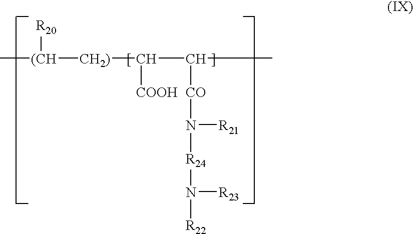

in which R₂₀ represents a hydrogen atom or a CH₃O, CH₃CH₂O or phenyl group, R₂₁ denotes a hydrogen atom or a lower alkyl group, such as methyl or ethyl, R₂₂ denotes a hydrogen atom or a lower C₁-C₆ alkyl group, such as methyl or ethyl, and R₂₃ denotes a lower C₁-C₆ alkyl group, such as methyl or ethyl, or a group corresponding to the formula: —R₂₄—N(R₂₂)₂, R₂₄ representing a —CH₂—CH₂—, —CH₂—CH₂—CH₂— or —CH₂—CH(CH₃)— group and R₂₂ having the meanings mentioned above.

(7) Polymers derived from the N-carboxyalkylation of chitosan, such as N-(carboxymethyl)chitosan or N-(carboxybutyl)chitosan.

(8) Amphoteric polymers of the -D-X-D-X— type chosen from:

-D-X-D-X-D- (X)

where D denotes a group

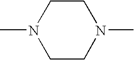

and X denotes the symbol E or E′, E or E′, which may be identical or different, denoting a divalent group which is a straight- or branched-chain alkylene group comprising up to 7 carbon atoms in the main chain which is unsubstituted or substituted by hydroxyl groups and which can additionally comprise oxygen, nitrogen or sulphur atoms or 1 to 3 aromatic and/or heterocyclic rings; the oxygen, nitrogen and sulphur atoms being present in the form of ether, thioether, sulphoxide, sulphone, sulphonium, alkylamine or alkenylamine groups or hydroxyl, benzylamine, amine oxide, quaternary ammonium, amide, imide, alcohol, ester and/or urethane groups.

b) polymers of formula:

-D-X-D-X— (X′)

where D denotes a group

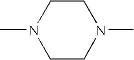

and X denotes the symbol E or E′ and at least once E′, E having the meaning indicated above and E′ being a divalent group which is a straight- or branched-chain alkylene group having up to 7 carbon atoms in the main chain which is unsubstituted or substituted by one or more hydroxyl groups and which comprises one or more nitrogen atoms, the nitrogen atom being substituted by an alkyl chain optionally interrupted by an oxygen atom and necessarily comprising one or more carboxyl functional groups and one or more hydroxyl functional groups and betainized by reaction with chloroacetic acid or sodium chloroacetate.

(9) (C₁-C₅)Alkyl vinyl ether/maleic anhydride copolymers partially modified by semiamidation with an N,N-dialkylaminoalkylamine, such as N,N-dimethylaminopropylamine, or by semiesterification with an N,N-dialkylaminoalkanol. These copolymers can also comprise other vinyl comonomers, such as vinylcaprolactam.

Mention will be made, among the amphoteric fixing polymers mentioned above which are the most particularly preferred according to the invention, of those of family (3), such as the copolymers with the CTFA name of octylacrylamide/acrylates/butylaminoethyl methacrylate copolymer, such as the products sold under the names Amphomer®, Amphomer® LV 71 or Lovocryl® 47 by National Starch, and those of family (4), such as methyl methacrylate/methyl dimethylcarboxymethylammonioethyl methacrylate copolymers.

The non-ionic fixing polymers which can be used according to the present invention are chosen, for example, from:
polyalkyloxazolines;
vinyl acetate homopolymers;
copolymers of vinyl acetate and of acrylic ester;
copolymers of vinyl acetate and of ethylene;
copolymers of vinyl acetate and of maleic ester, for example of dibutyl maleate;
acrylic ester copolymers, such as, for example, copolymers of alkyl acrylates and of alkyl methacrylates, such as the products provided by Röhm & Haas under the names Primal® AC-261 K and Eudragit® NE 30 D, by BASF under the name 8845 or by Hoechst under the name Appretan® N9212;
copolymers of acrylonitrile and of a non-ionic monomer chosen, for example, from butadiene and alkyl(meth)acrylates; mention may be made of the products provided under the name CJ 0601 B by Röhm & Haas;
styrene homopolymers;
copolymers of styrene and of alkyl(meth)acrylate, such as the products Mowilith® LDM 6911, Mowilith® DM 611 and Mowilith® LDM 6070 provided by Hoechst or the products Rhodopas® SD 215 and Rhodopas® DS 910 provided by Rhodia Chimie;
copolymers of styrene, of alkyl methacrylate and of alkyl acrylate;
copolymers of styrene and of butadiene;
copolymers of styrene, of butadiene and of vinylpyridine;
copolymers of alkyl acrylate and of urethane;
polyamides,

vinyllactam homopolymers and copolymers.

The alkyl groups of the non-ionic polymers mentioned above preferably have from 1 to 6 carbon atoms.

According to the present invention, the non-ionic fixing polymers possessing vinyllactam units can be those described in U.S. Pat. No. 3,770,683, U.S. Pat. No. 3,929,735, U.S. Pat. No. 4,521,504, U.S. Pat. No. 5,158,762 and U.S. Pat. No. 5,506,315 and in Patent Applications WO 94/121148, WO 96/06592 and WO 96/10593. They can be provided in the pulverulent form or in the form of a solution or suspension.

Homopolymers or copolymers possessing vinyllactam units comprise units of formula (XI):

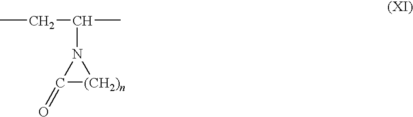

in which n is independently 3, 4 or 5.

The number-average molecular weight of the polymers possessing vinyllactam units is generally greater than approximately 5000, preferably between 10 000 and 1 000 000 approximately, more preferably between 10 000 and 100 000 approximately.

Mention may be made, among these fixing polymers, of polyvinylpyrrolidones, such as those sold under the name Luviskol® K30 by BASF; polyvinylcaprolactams, such as those sold under the name Luviskol® Plus by BASF; poly(vinylpyrrolidone/vinyl acetate) copolymers, such as those sold under the name PVPVA® S630L by ISP or Luviskol® VA 73, VA 64, VA 55, VA 37 and VA 28 by BASF; and poly(vinylpyrrolidone/vinyl acetate/vinyl propionate) terpolymers, such as, for example, those sold under the name Luviskol® VAP 343 by BASF.

The additional fixing polymers can also be chosen from optionally silicone-comprising polyurethanes. Mention may be made, as fixing polymers of polyurethane type, of the polymers Luviset PUR and Luviset Si PUR provided by BASF.

These additional fixing polymers can also be chosen from grafted silicones comprising a hydrocarbon backbone and silicone grafts or comprising a silicone backbone and hydrocarbon grafts, such as the products VS 70 and VS 80 provided by 3M. They can also be chosen from polymers comprising sulpho groups, such as the AQ resins (AQ55, AQ38 and AQ48) provided by Eastman Chemical.

The composition according to the invention can then comprise at least 0.01% by weight of additional fixing polymer, with respect to the total weight of the composition. More preferably, the composition according to the invention comprises from 0.01 to 20% by weight of additional fixing polymer, better still from 0.05 to 15% by weight and more preferably still from 0.1 to 10% by weight, with respect to the total weight of the composition.

The compositions according to the invention can also comprise at least one thickener chosen from polymeric thickeners which are natural or synthetic, anionic, amphoteric, zwitterionic, non-ionic or cationic and associative or nonassociative, and nonpolymeric thickeners, such as, for example, an electrolyte or a sugar.

The compositions in accordance with the invention can be packaged in a pot, in a tube, in a pump-action spray or in an aerosol device conventional in cosmetics.

The compositions according to the invention can, when they are intended to be packaged in a device of aerosol type, comprise at least one propellant, such as a propellant gas.

The propellant gas can then be chosen, for example, from dimethyl ether, C₃ to C₅ alkanes, halogenated hydrocarbons and their mixtures.

The compositions according to the invention can additionally comprise at least one additive chosen from pearlescent agents; opacifying agents; plasticizing agents; sunscreens; fragrances; colorants, preservatives; pH-stabilizing agents; acids; bases; polyols (for example glycols); inorganic fillers; glitter, and any other additive conventionally used in the cosmetics field.

A person skilled in the art will take care to choose the optional additives and their amounts so that they do not interfere with the properties of the compositions of the present invention.

These additives are present in the composition according to the invention in an amount ranging from 0 to 50% by weight, with respect to the total weight of the composition.

The compositions according to the invention can be provided in the form of more or less thickened liquids, of gels, of creams, of pastes or of foams.

Preferably, they are provided in the form of gels.

The composition according to the invention can advantageously be used as a hair product. It can in particular be used as a styling product, for example as a styling gel.

According to a preferred embodiment, it is used as leave-in styling product, that is to say for the shaping and/or form retention of the hairstyle, not followed by rinsing.

The present invention also relates to a method for the cosmetic treatment of the hair, for example a hair care method, or a method for the shaping and/or form retention of the hairstyle, which consists in applying, to the hair, an effective amount of a composition as defined above and then carrying out an optional rinsing after an optional leave-in time.

Preferably, the composition according to the invention is not rinsed out.

The following example is given by way of illustration of the present invention.

### EXAMPLE

In the following example, all the amounts are indicated as percent by weight of active material (AM), with respect to the total weight of the composition, unless otherwise indicated.

This example illustrates the formulation of a styling gel in accordance with the invention.

Such a gel was prepared from the ingredients shown in the table below:

| Ingredients | Contents |
|---|---|
| (Meth)acrylates/hydroxyester of (meth)acrylates | 3% |
| copolymer (1) | |
| Crosslinked poly(2-acrylamido-2- | 2% |
| methylpropanesulphonic acid) neutralized with | |
| ammonia (2) | |
| Glycerol | 3% |
| Preservatives, neutralizing agents, fragrances | q.s. |
| Water | q.s. for 100% |

(1) Sold under the name Acudyne DHR by Röhm & Haas.
(2) Sold under the name Hostacerin AMPS by Clariant.

### Results Obtained:

The performance of the styling gel composition described above was evaluated by professionals on panels of models.

This composition makes it possible to obtain very good fixing of the hair which is both flexible and lasting. In particular, the fixing polymer film shows very little friability.

In addition, this composition has proved to provide excellent cosmetic properties to the hair. In particular, after disentangling, the hair is not dry and is particularly soft.

1-43. (canceled)

44. A cosmetic composition comprising, in a cosmetically acceptable medium:
at least one copolymer of acrylate(s) or of methacrylate(s) and of hydroxyester of acrylate(s) or of hydroxyester of methacrylate(s), and
at least one partially or completely neutralized sulphonated polymer comprising at least one unit derived from a monomer comprising ethylenic unsaturation and comprising a sulpho group,
wherein the weight ratio of (the amount of the at least one copolymer of acrylate(s) or of methacrylate(s) and of hydroxyester of acrylate(s) or of hydroxyester of methacrylate(s)) to (the amount of the at least one partially or completely neutralized sulphonated polymer(s)), has a value ranging from 0.1 to 10.

45. A cosmetic composition according to claim 44, wherein the at least one copolymer of acrylate(s) or of methacrylate(s) and of hydroxyester of acrylate(s) or of hydroxyester of methacrylate(s) results from the copolymerization of at least two monomers, wherein:
the at least one first monomer is chosen from acrylic acid, methacrylic acid, C1 to C4 alkyl acrylates and C1 to C4 alkyl methacrylates, and
the at least one second monomer is chosen from C1 to C4 hydroxyalkyl acrylates and C1 to C4 hydroxyalkyl methylacrylates.

46. A cosmetic composition according to claim 45, wherein the at least one copolymer of acrylate(s) or of methacrylate(s) and of hydroxyester of acrylate(s) or of hydroxyester of methacrylate(s) results from the copolymerization of at least three monomers, wherein:
the at least one first monomer is chosen from acrylic acid and methacrylic acid,
the at least one second monomer is chosen from C1 to C4 alkyl acrylates and C1 to C4 alkyl methacrylates, and
the at least one third monomer is chosen from C1 to C4 hydroxyalkyl acrylates and C1 to C4 hydroxyalkyl methacrylates.

47. A cosmetic composition according to claim 46, wherein the at least one second monomer comprises at least one C1 or C2 alkyl acrylate or C1 or C2 alkyl methacrylate.

48. A cosmetic composition according to claim 46, wherein the at least one third monomer is chosen from hydroxyethyl acrylate and hydroxyethyl methacrylate.

49. A cosmetic composition according to claim 44, wherein the cosmetic composition comprises a blend of at least two copolymers of acrylate(s) or of methacrylate(s) and of hydroxyester of acrylate(s) or of hydroxyester of methacrylate(s).

50. A cosmetic composition according to claim 49, wherein the at least two copolymers of acrylate(s) or of methacrylate(s) and of hydroxyester of acrylate(s) or of hydroxyester of methacrylate(s) each result from the copolymerization of at least three monomers, wherein:
the at least one first monomer is chosen from acrylic acid and methacrylic acid,
the at least one second monomer is chosen from C1 to C4 alkyl acrylates and C1 to C4 alkyl methacrylates, and
the at least one third monomer is chosen from C1 to C4 hydroxyalkyl acrylates and C1 to C4 hydroxyalkyl methacrylates.

51. A cosmetic composition according to claim 49, wherein the blend of at least two copolymers of acrylate(s) or of methacrylate(s) and of hydroxyester of acrylate(s) or of hydroxyester of methacrylate(s) comprises a first copolymer resulting from the copolymerization of at least three monomers, wherein:
the at least one first monomer is chosen from acrylic acid and methacrylic acid,
the at least one second monomer is chosen from C1 to C4 alkyl acrylates and C1 to C4 alkyl methacrylates, and
the at least one third monomer is chosen from C1 to C4 hydroxyalkyl acrylates and C1 to C4 hydroxyalkyl methacrylates;
and a second copolymer resulting from the copolymerization of at least two monomers, wherein:
the at least one first monomer is chosen from C1 to C4 alkyl acrylates and C1 to C4 alkyl methacrylates, and
the at least one second monomer is chosen from C1 to C4 hydroxyalkyl acrylates and C1 to C4 hydroxyalkyl methacrylates;
wherein the second copolymer does not comprise monomers composed of unsaturated carboxylic acids.

52. A cosmetic composition according to claim 49, wherein the at least two copolymers of the blend have different glass transition temperatures.

53. A cosmetic composition according to claim 52, wherein the difference in glass transition temperature between the at least two copolymers is greater than or equal to 10° C.

54. A cosmetic composition according to claim 44, wherein the cosmetic composition comprises at least 0.01% by weight of the at least one copolymer of acrylate(s) or of methacrylate(s) and of hydroxyester of acrylate(s) or of hydroxyester of methacrylate(s), with respect to the total weight of the composition.

55. A cosmetic composition according to claim 54, wherein the cosmetic composition comprises from 0.01 to 20% by weight of the at least one copolymer of acrylate(s) or of methacrylate(s) and of hydroxyester of acrylate(s) or of hydroxyester of methacrylate(s), with respect to the total weight of the composition.

56. A cosmetic composition according to claim 44, wherein the sulphonated polymer comprises at least one monomer comprising ethylenic unsaturation and comprising a sulpho group chosen from vinylsulphonic acid, styrenesulfonic acid, (meth)acrylamido(C1-C22)alkylsulphonic acids, N—(C1-C22)alkyl(meth)acrylamido(C1-C22)alkylsulphonic acids, and the partially or completely neutralized forms thereof.

57. A cosmetic composition according to claim 56, wherein the at least one partially or completely neutralized sulphonated polymer comprises at least one monomer comprising ethylenic unsaturation chosen from (meth)acrylamido(C1-C22)alkylsulphonic acids and their partially or completely neutralized forms.

58. A cosmetic composition according to claim 57, wherein the at least one partially or completely neutralized sulphonated polymer comprises 2-acrylamido-2-methylpropanesulphonic acid (AMPS) and/or its partially or completely neutralized forms.

59. A cosmetic composition according to claim 44, wherein the at least one partially or completely neutralized sulphonated polymer is crosslinked.

60. A cosmetic composition according to claim 44, wherein the at least one partially or completely neutralized sulphonated polymer is partially or completely neutralized with an inorganic base and/or an organic base.

61. A cosmetic composition according to claim 60, wherein the at least one partially or completely neutralized sulphonated polymer comprises at least 90% of sulpho groups in the salified form.

62. A cosmetic composition according to claim 61, wherein the at least one partially or completely neutralized sulphonated polymer is a crosslinked poly(2-acrylamido-2-methylpropanesulphonic acid) neutralized at least at 90% comprising:
(a) from 90 to 99.9% by weight of units of following general formula (A): 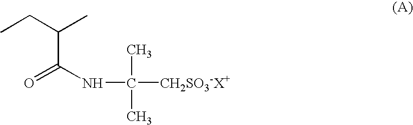
in which X⁺ is a proton, an alkali metal cation, an alkaline earth metal cation, or the ammonium ion;
(b) from 0.01 to 10% by weight of crosslinking units originating from at least one monomer having at least two olefinic double bonds; wherein the weight percent is defined with respect to the total weight of the at least one partially or completely neutralized sulphonated polymer.

63. A cosmetic composition according to claim 62, wherein the crosslinking units originating from at least one monomer having at least two olefinic double bonds are chosen from divinylbenzene, diallyl ether, dipropylene glycol diallyl ether, polyglycol diallyl ethers, triethylene glycol divinyl ether, hydroquinone diallyl ether, ethylene glycol di(meth)acrylate, tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl(meth)acrylate, allyl ethers of alcohols of the series of the sugars, or other allyl or vinyl ethers of polyfunctional alcohols, and allyl esters of phosphoric and/or vinylphosphonic acid derivatives, and mixtures thereof.

64. A cosmetic composition according to claim 56, wherein the at least one partially or completely neutralized sulphonated polymer further comprises at least one unit derived from hydrophilic monomers comprising ethylenic unsaturation and not comprising a fatty chain chosen, for example, from (meth)acrylic acids, their alkyl derivatives substituted at the β position, or their esters obtained with monoalcohols or with mono- or polyalkylene glycols, (meth)acrylamides, vinylpyrrolidone, maleic anhydride, itaconic acid, and maleic acid.

65. A cosmetic composition according to claim 56, wherein the at least one partially or completely neutralized sulphonated polymer is chosen from amphiphilic copolymers of AMPS and of at least one hydrophobic monomer comprising ethylenic unsaturation, and comprising at least one hydrophobic part having from 6 to 50 carbon atoms.

66. A cosmetic composition according to claim 65, wherein the at least one hydrophobic monomer comprising ethylenic unsaturation is chosen from acrylates and acrylamides of following formula (B): 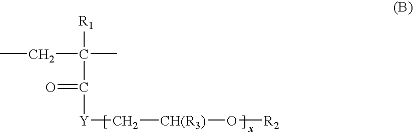
in which R1 and R3, which are identical or different, are a hydrogen atom or a linear or branched C1-C6 alkyl radical; Y is O or NH; R2 is a hydrophobic hydrocarbon radical comprising from 6 to 50 carbon atoms; and X is the number of moles of alkylene oxide and ranges from 0 to 100.

67. A cosmetic composition according to claim 44, wherein the cosmetic composition comprises at least 0.01% by weight of the at least one partially or completely neutralized sulphonated polymer, with respect to the total weight of the composition.

68. A cosmetic composition according to claim 67, wherein the cosmetic composition comprises from 0.01% to 10% by weight of the at least one partially or completely neutralized sulphonated polymer, with respect to the total weight of the composition.

69. A cosmetic composition according to claim 44, wherein the weight ratio of (the amount of the at least one copolymer(s) of acrylate(s) or of methacrylate(s) and of hydroxyester of acrylate(s) or of hydroxyester of methacrylate(s)) to (the amount of the at least one partially or completely neutralized sulphonated polymer(s)) has a value ranging from 1 to 8.

70. A cosmetic composition according to claim 44, wherein the cosmetically acceptable medium comprises water and/or at least one cosmetically acceptable solvent chosen from lower C1-C4 alcohols; polyols; polyol ethers; C5-C10 alkanes; C3-4 ketones; C1-C4 alkyl acetates; dimethoxyethane, and diethoxyethane

71. A cosmetic composition according to claim 44, wherein the cosmetic composition further comprises at least one fatty substance chosen from plant oils, animal oils, mineral oils, natural or synthetic oils, fatty alcohols, and waxes.

72. A cosmetic composition according to claim 71, wherein the at least one fatty substance is present in an amount ranging from 0.1 to 30% by weight, with respect to the total weight of the composition.

73. A cosmetic composition according to claim 44, wherein the cosmetic composition further comprises at least one surfactant chosen from cationic surfactants, anionic surfactants, non-ionic surfactants, and amphoteric and zwitterionic surfactants.

74. A cosmetic c according to claim 73, wherein the cosmetic composition comprises at least 0.01% by weight of the at least one surfactant, with respect to the total weight of the composition.

75. A cosmetic composition according to claim 74, wherein the cosmetic composition comprises from 0.01 to 20% by weight of the at least one surfactant, with respect to the total weight of the composition.

76. A cosmetic composition according to claim 44, wherein the cosmetic composition further comprises at least one entity chosen from silicone and silicone derivatives in the soluble, dispersed, or microdispersed form.

77. A cosmetic c according to claim 44, wherein the cosmetic composition further comprises at least one additional fixing polymer chosen from those of anionic, cationic, non-ionic, and amphoteric nature, other than the at least one copolymer of acrylate(s) or of methacrylate(s) and of hydroxyester of acrylate(s) or of hydroxyester of methacrylate(s).

78. A cosmetic composition according to claim 77, wherein the cosmetic composition comprises at least 0.01% by weight of the at least one additional fixing polymer, with respect to the total weight of the composition.

79. A cosmetic composition according to claim 78, wherein the cosmetic composition comprises from 0.01 to 20% by weight of the at least one additional fixing polymer, with respect to the total weight of the composition.

80. A cosmetic composition according to claim 44, further comprising at least one additive chosen from pearlescent agents; opacifying agents; plasticizing agents; sunscreens; fragrances; colorants, preservatives; pH-stabilizing agents; acids; bases; polyols; inorganic fillers; glitter, and any other additive conventionally used in the cosmetics field.

81. A cosmetic composition according to claim 44, wherein the cosmetic composition is in the form of a hair product chosen from a more or less thickened liquid, a hair gel, a cream, a paste, or a foam.

82. A cosmetic composition according to claim 81, wherein the cosmetic composition is a hair product in the form of a hair gel.

83. A method for the cosmetic treatment of the hair, comprising applying, to the hair, an effective amount of a cosmetic composition according to claim 44, and optionally rinsing after an optional leave-in time.

84. A method according to claim 83, wherein the cosmetic composition is not rinsed out.

85. A method for the shaping and/or the form retention of a hairstyle, comprising applying, to the hair, an effective amount of a cosmetic composition according to claim 44, and optionally rinsing after an optional leave-in time.
